(19) United States
(12) Patent Application Publication
(10) Pub. No.: US 2008/0015668 A1
(43) Pub. Date: Jan. 17, 2008

(54) Test Connector for Implantable Leads

(76) Inventor: Thomas M. Soukup, Maple Grove, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/590,369

(22) PCT Filed: Feb. 23, 2005
(86) PCT No.: PCT/US05/05761

§ 371 Date: May 17, 2007

Related U.S. Application Data
(60) Provisional application No. 60/546,791, filed on Feb. 23, 2004.

(51) Int. Cl. A61N 1/05

(52) U.S. Cl. 607/115

(57) ABSTRACT

Testing connectors for use with implantable leads are disclosed. The testing connectors are suitable for attachment to an implantable medical lead connector to facilitate electrical testing of the implantable lead. In some cases, a distal end of the lead can be repositioned within a body while the lead is electrically connected to a test device. A testing connector comprising the housing having a first side (106) and a second side (108) is disclosed. The first side of the housing defines a first channel and the second side of the housing defines a second channel. The first channel and the second channel are dimensioned to receive at least a portion of an implantable lead.

### FIELD OF THE INVENTION

The present invention relates generally to test connectors for implantable leads and the like. More particularly, the present invention relates to testing connectors suitable for attachment to an implantable medical lead connector to facilitate electrical testing of the implantable lead.

### BACKGROUND OF THE INVENTION

Implantable medical devices, such as pulse generators, generally have at least one implantable lead that connects the device to a patient's heart. Typically, an implantable lead has a proximal portion including a connector adapted to be inserted within a corresponding port of the implantable device. The connector of the implantable lead can comprise one or more conductive interfaces on the exterior surface of the connector for making suitable connection to the contacts located within a corresponding port of the implantable device. Implantable devices and implantable leads are generally described in, for example, U.S. Pat. No. 6,321,126 to Kuzma, entitled “Implantable Connector,” and U.S. Pat. No. 5,086,773 to Ware, entitled “Tool-less Pacemaker Lead Assembly,” which are both incorporated herein by reference.

Generally, when an implantable medical device is placed into a human patient, testing procedures are conducted in order to determine, for example, suitable placement of the implantable lead, minimum defibrillation threshold, stimulation pulse output energy, lead conductivity, and electrode integrity among other things. In some procedures, the implantable lead is advanced into the patient's heart through a vein by a stylet or other suitable device. Once the distal end of lead contacts the heart, the physician generally tests the implantable lead to determine if the lead placement is acceptable, before connecting the proximal end of the lead to the implantable device. The testing of the lead can involve attaching the connectors on the proximal end of the implantable lead to an analyzer. During the testing procedure, it may be necessary for the physician to move the distal end of the lead by advancing or retracting the stylet, in order to locate an acceptable site for the placement of the implantable lead.

The above-mentioned tests associated with implantable leads are typically conducted in the operating room during the implant procedure. Consequently, several issues can arise with respect to the testing connector used to couple the implantable lead connector to an analyzer including, for example, damage to the connector leads and two hand attachment of the testing connector to the implantable lead. Damage to the connector leads can increase the time and expense of the implant procedure, since a new lead may need to be used and routed inside the patient to a suitable location in the heart. Additionally, the physician will generally have one hand on the stylet or other actuating device connected to the lead during the procedure, and therefore may not have both hands available for operating and/or attaching a testing connector.

Due to the increasing number of medical procedures and treatment strategies employing implantable devices, it would be desirable to provide a testing connector for implantable medical leads that could address all of the above-mentioned limitations.

### SUMMARY OF THE INVENTION

In a first aspect, the invention pertains to an apparatus for testing an implantable medical lead. The implantable medical lead has a proximal portion including a connector adapted to be inserted within a corresponding port of a medical device. The connector includes a plurality of conductive interfaces on an exterior surface of the connector. The testing apparatus comprises a handheld housing structure having a channel adapted to receive at least a portion of a connector of the implantable lead. In this embodiment, the testing apparatus further comprises one or more electrically conductive contact members positioned in a mating orientation with at least a portion of the conductive interfaces, such that the electrically conductive contact members can contact at least a portion of the conductive interfaces on the connector when the connector is positioned in the channel.

In a further aspect, the invention pertains to a method for testing an implantable lead. The method comprises establishing electrical connection with a portion of the conductive interfaces on a connector by positioning the connector in a testing apparatus, wherein the testing apparatus comprises a channel with one or more electrically conductive contact member positioned within the channel in a mating orientation with at least a portion of the conductive interfaces.

### BRIEF DESCRIPTION OF THE FIGURES

FIG. 1 is a plan view showing an implantable lead and a testing connector in accordance with an exemplary embodiment of the present invention.

FIG. 2 is an isometric view of the testing connector shown in the previous figure.

FIG. 3 is an additional isometric view of the testing connector shown in the previous figure.

FIG. 4 is a plan view showing the testing connector shown in the previous figure.

FIG. 5 is an additional plan view showing the testing connector of the previous figure.

FIG. 6 is a plan view showing a testing connector in accordance with an exemplary embodiment of the present invention.

FIG. 7 is an isometric view of the testing connector shown in the previous figure.

FIG. 8 is an additional isometric view of the testing connector shown in the previous figure.

FIG. 9 is a plan view showing the testing connector shown in the previous figure.

FIG. 10 is an axial plan view showing a testing connector in accordance with an additional exemplary embodiment of the present invention.

FIG. 11 is a cross-sectional view of the testing connector shown in the previous figure.

FIG. 12 is an additional axial view of the testing connector shown in the previous figure.

FIG. 13 is a side view including the testing connector shown in the previous figure. In the embodiment of FIG. 13, a hand is disposed about a housing of the test connector so that the housing is received in the palm of the hand.

FIG. 14 is an axial plan view showing a testing connector in accordance with an yet another exemplary embodiment of the present invention.

FIG. 15 is a cross-sectional view of an assembly including a testing connector and an implantable lead.

### DETAILED DESCRIPTION OF THE INVENTION

Improved testing connectors comprise a handheld housing having a channel adapted to receive and hold the proximal end of an implantable lead. Due to the presence of the channel, the testing connectors can be coupled and uncoupled from an appropriate connector on the implantable lead without damaging the connector. The testing connectors further comprise one or more electrically conductive contact member positioned in a mating orientation with at least one of the conductive interfaces located on the connector of an implantable lead. Generally, the mating orientation is established by inserting the connector portion of an implantable lead into the channel of the screening connector. In some embodiments, the channel is adapted to receive and hold an IS-1 connector, while in other embodiments the channel is adapted to receive and hold an IS-4 connector.

As noted above, implantable leads associated with implantable devices are generally tested prior to final placement of the implantable lead within the patient. Since the testing procedure generally occurs in the operating room during a surgical procedure to implant the device and associated leads, damage to the connector during the testing procedure can increase the expense and time to complete the procedure. Additionally, the physician will generally have one hand on a stylet or other suitable device for changing the position of the distal end of the implantable lead. As a result, it is desirable in some applications to provide a handheld testing connector that can be coupled and uncoupled to a connector of an implantable lead with only one hand, and which will not damage the connector during the coupling/uncoupling process. As described herein, damage to the connector of an implantable lead can be prevented by employing a handheld connector with a channel adapted to receive a specific connector structure, such as an IS-1 connector. Additionally, the channel can allow a physician to couple and uncouple the testing connector from the connector using only one hand.

The testing connectors of the present invention generally comprise a channel or groove that is adapted to hold and receive the connector portion of an implantable lead. In some embodiments, the channel can be designed to receive an IS-1 connector, while in other embodiments the channel can be designed to receive an IS-4 connector. In some embodiments, the testing connector may comprise a unitary structure for the handheld housing with an opening at one end, while in other embodiments the testing connector can comprise a plurality of component pieces that form a handheld housing. The testing connectors can further comprise one or more electrically conductive contact members positioned in a mating orientation with a portion of the electrical interfaces located along an exterior surface of a connector positioned within the channel.

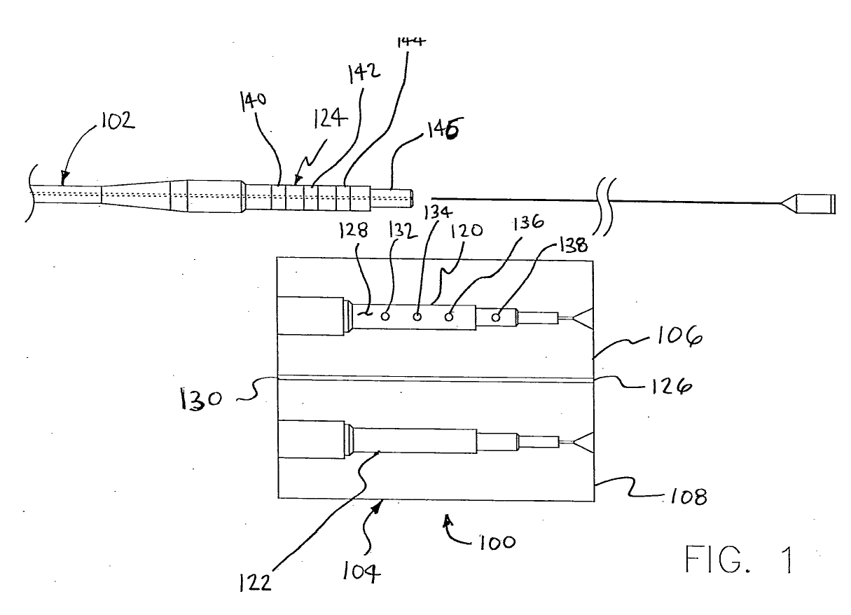

FIG. 1 is a plan view showing an implantable lead 102 and a testing connector 100 in accordance with an exemplary embodiment of the present invention. Testing connector 100 is shown comprising a handheld housing 104 having a first side 106 and a second side 108. As shown in FIG. 1, first side 106 defines a first channel 120 and second side 108 defines a second channel 122. In the embodiment of FIG. 1, first channel 120 and second channel 122 are generally complimentary structures that are adapted to enclose a connector 124 of implantable lead 102, for example, when first side 106 and second side 108 assume a closed configuration. First side 106 and second side 108 may, for example, define a cavity 128 that is dimensioned to receive connector 124 when first side 106 and second side 108 assume a closed configuration. Those of skill in the art will recognize that connector 124 of implantable lead 102 may comprise various connectors without deviating from the spirit and scope of the present invention. Examples of connectors that may be suitable in some applications include IS-1 connectors and IS-4 connectors.

In some embodiments of the present invention, first side 106 can be hingedly connected to second side 108 such that second side 108 can be rotated relative to first side to enclose connector 124 of implantable lead 102. In the embodiment of FIG. 1, first side 106 is connected to second side 108 by a hinge 126 comprising a web 130. In some embodiments, second side 108 can be rotated up about 180 degrees relative to first side 106, while in other embodiments second side 108 may be rotated from about 50 degrees to about 150 degrees relative to first side 106. Those of skill in the are will recognize that various ranges of rotation one side relative to the other side can be used without deviating from the spirit and scope of the present invention.

In some embodiments, second side 108 can further comprise a latch member, which can couple with a corresponding structure located on first side 106 to secure second side 108 to first side 106. The latch member and corresponding structure can be any mechanical system capable of coupling the second side of the testing connector to the first side of the connector. In some embodiments, the latch structure can be operated using only one hand, which permits a physician or other operator to have a free hand available during attachment of the testing connector to a connector. In some embodiments, the latch member and corresponding structure can comprise, for example, a slot and protrusion mechanism or the like. One of ordinary skill in the art will recognize that additional latch and corresponding structures are contemplated and are within the scope of the present disclosure.

Referring still to FIG. 1, as describe previously, testing connector 100 can comprise a first side 106 and second side 108 with a first channel 120 and a second channel 122 formed into each side, respectively. Due to the channels formed into the testing connector, the proximal end of implantable lead 102 can be enclosed within and/or removed from the testing connector without damaging the connector portion of the implantable lead. Additionally, the handheld design of the housing preferably permits a physician to attach testing connector 100 to an implantable lead using only one hand, which permits the physician to have a free hand to, for example, adjust the position of the distal end of the implantable lead using a stylet. Additionally, the housing and a latch may be dimensioned to permit the physician to detach the testing connector from the implantable lead using only one hand.

In the embodiment of FIG. 1, testing connector 100 comprises a first contact 132, a second contact 134, a third contact 136 and a fourth contact 138. Also in the embodiment of FIG. 1, connector 124 of implantable lead 102 comprises a first conductive ring 140, a second conductive ring 142, a third conductive ring 144, and a fourth conductive ring 146. The contacts may be positioned in a mating orientation relative to the conductive rings of connector 124. Generally, the mating orientation is established when a connector of an implantable lead is positioned within an appropriate testing connector. As shown in FIG. 1, the contacts are positioned such that when connector 124 is positioned in first channel 120, the contacts can contact at least a portion of the conductive rings on connector 124.

In some embodiments, the contact extend through holes in first side 106 such that a portion of the contacts are exposed on the exterior surface of the testing connector 100 and are available for further connection. The contacts can be connected to an analyzer or other test device with, for example, alligator clips, wires or the like. The number of contacts used in a particular testing connector can be varied without deviating from the spirit and scope of the present invention. Additionally, the spacing of the contacts may generally be guided by the design of the connector that the testing connector will interface with.

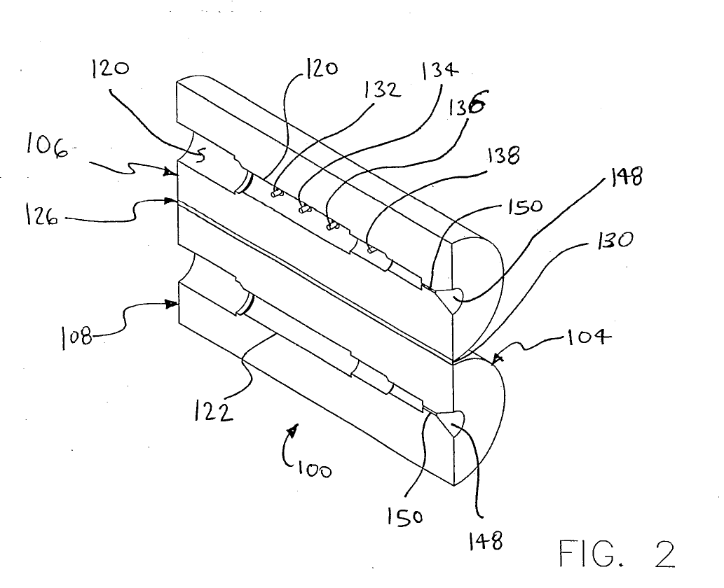

FIG. 2 is an isometric view of testing connector 100 shown in the previous figure. Testing connector 100 comprises a housing 104 comprising a first side 106 that is connected to a second side 108 by a hinge 126. In the embodiment of FIG. 2, hinge 126 comprises a web 130.

In the embodiment of FIG. 2, first side 106 of housing 104 defines a first channel 120. With reference to FIG. 2, it will be appreciated that first side 106 of housing 104 supports a first contact 132, a second contact 134, a third contact 136 and a fourth contact 138. In FIG. 2, the contacts can be seen extending into first channel 120 defined by first side 106. The contacts may, for example, comprise spring-loaded contacts. Spring-loaded contacts that may be suitable in some applications are commercially available from Mill-Max Manufacturing Corporation of Oyster Bay, N.Y.

With reference to FIG. 2, it will be appreciated that second side 108 of housing 104 defines a second channel 122. With continuing reference to FIG. 2, it will be appreciated that first channel 120 and second channel 122 each include a tapered portion 148 and a guiding portion 150. In some embodiments of the present invention, each guiding portion 150 has a diameter that is similar an outer diameter of a stylet. When this is the case, guiding portion 150 may guide the stylet into the lumen of a lead. In some embodiments of the present invention, each tapered portion 148 is shaped so as to funnel the distal end of a stylet into guiding portion 150. When this is the case, tapered portion 148 and guiding portion 150 may facilitate insertion of the stylet into the lumen of a lead.

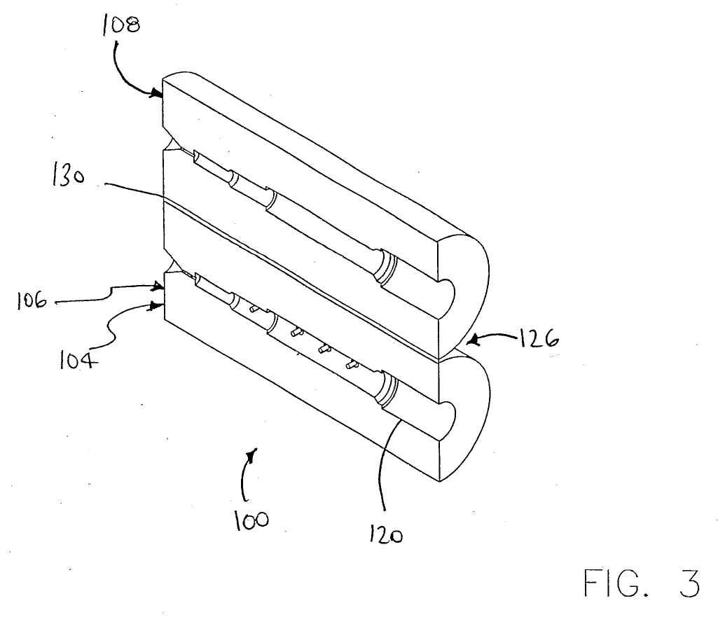

FIG. 3 is an additional isometric view of testing connector 100 shown in the previous figure. Testing connector 100 comprises a housing 104 including a first side 106 and a second side 108. In FIG. 3, first side 106 and second side 108 are hingingly coupled to one another by a hinge 126. In the embodiment of FIG. 3, hinge 126 comprises a web 130. First side 106 of housing 104 defines a first channel 120 and second side 108 defines a second channel 122. In some embodiments of the present invention, the first side 106 and the second side 108 can cooperate to define a cavity. In the embodiment of FIG. 3, first channel 120 and second channel 122 are shaped so that the cavity is capable of receiving a portion of a lead.

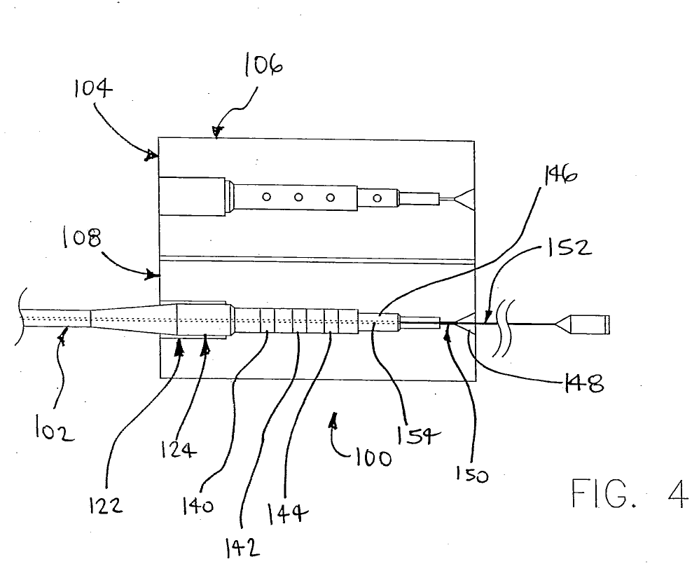

FIG. 4 is a plan view showing testing connector 100 shown in the previous figure. In the embodiment of FIG. 4, connector 124 of lead 102 is disposed in second channel 122 defined by second side 108 of housing 104. Stylet 152 can be seen extending from lumen 154 of lead 102. In FIG. 4, stylet 152 is shown extending through guiding portion 150 and tapered portion 148 of second channel 122.

Connector 124 of stylet 152 comprises a first conductive ring 140, a second conductive ring 142, a third conductive ring 144, and a fourth conductive ring 146. A first side 106 of housing 104 is connected to second side 108 by a hinge 126. First side 106 supports a first contact 132, a second contact 134, a third contact 136 and a fourth contact 138. With reference to FIG. 4, it will be appreciated that the positions of the contacts generally correspond with the positions of the conductive rings of connector 124. In the embodiment of FIG. 4, the contacts can be brought into contact with the conductive rings, for example by rotating first side 106 of housing 104 relative to second side 108.

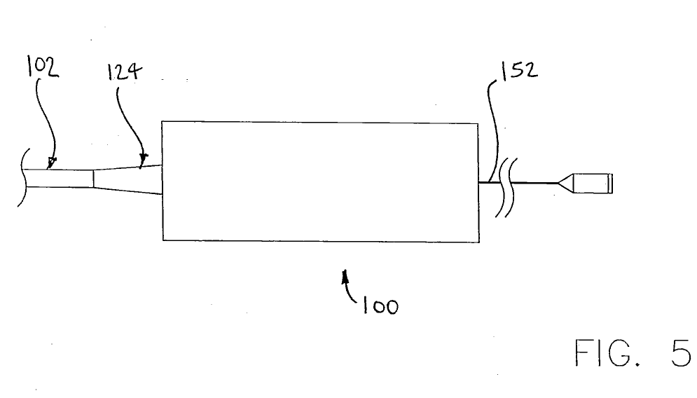

FIG. 5 is an additional plan view showing testing connector 100 of the previous figure. In the embodiment of FIG. 5, connector 124 of implantable lead 102 is shown extending into a cavity defined by testing connector 100. A portion of connector 124 of implantable lead 102 can be enclosed in testing connector 100, for example by rotating first side 106 relative second side 108. Stylet 152 can be seen extending from another end of testing connector 100 in FIG. 5.

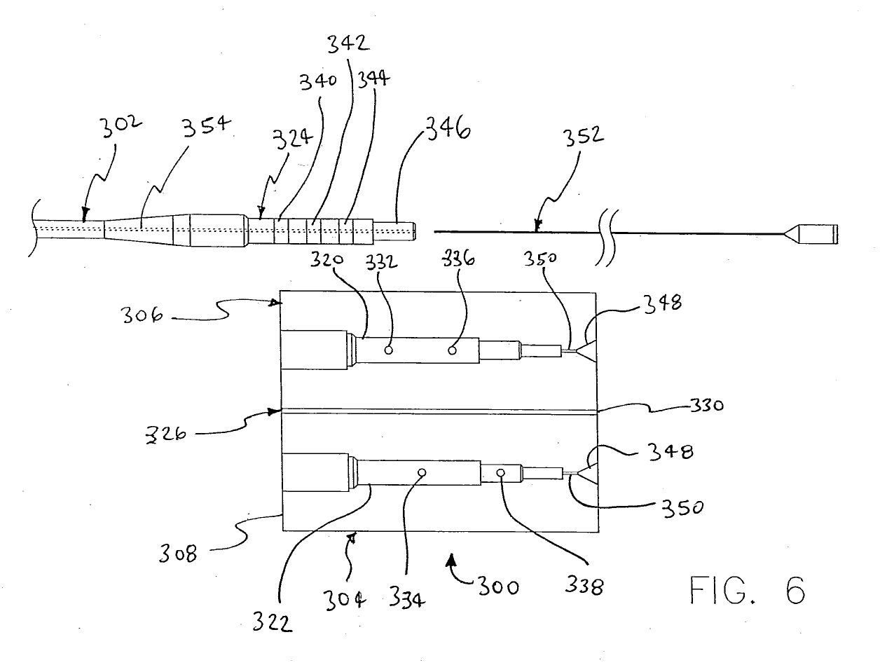

FIG. 6 is a plan view showing a testing connector 300 in accordance with an exemplary embodiment of the present invention. Testing connector 300 may used to facilitate the making of electrical connection with an implantable lead 302. Implantable lead 302 defines a lumen 354 that is dimensioned to receive a stylet 352. Stylet 352 may be used to reposition the distal end of lead 302 within the body of a patient.

In the embodiment of FIG. 6, lead 302 includes a connector 324 comprising a first conductive ring 340, a second conductive ring 342, a third conductive ring 344, and a fourth conductive ring 346. Testing connector 300 comprises a housing 304. Housing 304 comprises a first side 306 that is connected to a second side 308 by a hinge 326. In the embodiment of FIG. 6, hinge 326 comprises a web 330. First side 306 defines a first channel 320 and second side 308 defines a second channel 322. In the embodiment of FIG. 6, first channel 320 and second channel 322 are dimensioned so that they are capable of receiving a portion of connector 324 of lead 302.

With reference to FIG. 6, it will be appreciated that first side 306 supports a first contact 332 and a third contact 336. In the embodiment of FIG. 6, first contact 332 extends into first channel 320 and is positioned so as to contact first conductive ring 340 of connector 324 when connector 324 is placed in first channel 320. Also in the embodiment of FIG. 6, third contact 336 extends into first channel 320 and is positioned so as to contact third conductive ring 344 of connector 324 when connector 324 is placed in first channel 320.

With continuing reference to FIG. 6, it will be appreciated that second side 308 supports a second contact 334 and a fourth contact 338. In the embodiment of FIG. 6, second contact 334 extends into second channel 322 and is positioned so as to contact second conductive ring 342 of connector 324 when connector 324 is placed in second channel 322. Also in the embodiment of FIG. 6, fourth contact 338 extends into second channel 322 and is positioned so as to contact fourth conductive ring 346 of connector 324 when connector 324 is placed in second channel 322.

With reference to FIG. 6, it will be appreciated that first channel 320 and second channel 322 each include a tapered portion 348 and a guiding portion 350. In some embodiments of the present invention, each guiding portion 350 has a diameter that is similar an outer diameter of stylet 352. When this is the case, guiding portion 350 may guide stylet 352 into lumen 354 of lead 302. In some embodiments of the present invention, each tapered portion 348 is shaped so as to funnel the distal end of stylet 352 into guiding portion 350. When this is the case, tapered portion 348 and guiding portion 350 may facilitate insertion of stylet 352 into lumen 354 of lead 302.

With reference to FIG. 6, it will be appreciated that first channel 320 and second channel 322 are shaped so that connector 324 can be received between first side 306 and second side 308 while a portion of stylet 352 is extending from lumen 354 of lead 302. When this is the case, stylet 352 can be used to reposition the distal end of lead 302, while, at the same time, testing connector 300 is used to make a plurality of electrical connections with lead 302.

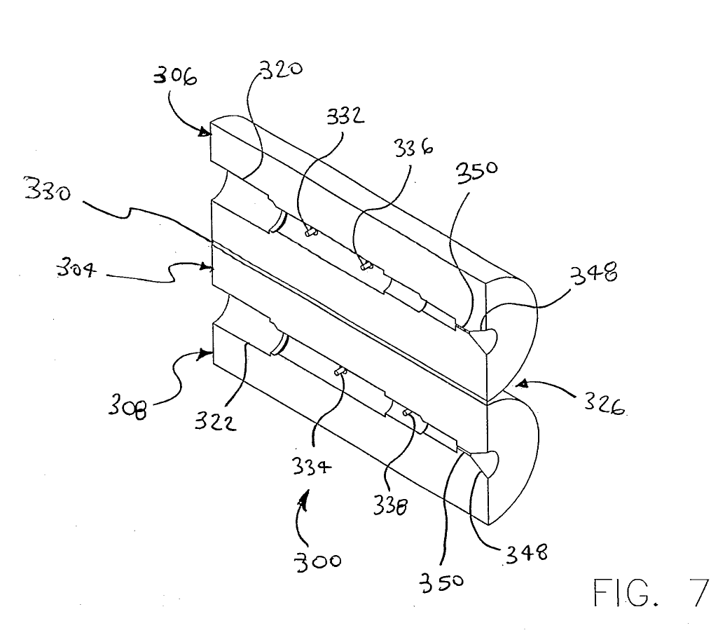

FIG. 7 is an isometric view of testing connector 300 shown in the previous figure. Testing connector 300 comprises a housing 304. Housing 304 comprises a first side 306 that is connected to a second side 308 by a hinge 326. In the embodiment of FIG. 7, hinge 326 comprises a web 330.

First side 306 of housing 304 defines a first channel 320. With reference to FIG. 7, it will be appreciated that first side 306 supports a first contact 332 and a third contact 336. In the embodiment of FIG. 7, first contact 332 and third contact 336 both extend into first channel 320. Second contact 334 and fourth contact 338 may comprise, for example, spring-loaded contacts. Spring-loaded contacts that may be suitable in some applications are commercially available from Mill-Max Manufacturing Corporation of Oyster Bay, N.Y.

With continuing reference to FIG. 7, it will be appreciated that second side 308 of housing 304 defines a second channel 322. Second side 308 supports a second contact 334 and a fourth contact 338 that are shown extending into second channel 322. In some embodiments of the present invention, second contact 334 and fourth contact 338 are biased to extend into second channel 322. For example, second contact 334 and fourth contact 338 may comprise spring-loaded contacts.

With reference to FIG. 7, it will be appreciated that first channel 320 and second channel 322 each include a tapered portion 348 and a guiding portion 350. In some embodiments of the present invention, each guiding portion 350 has a diameter that is similar an outer diameter of a stylet. When this is the case, guiding portion 350 may guide the stylet into the lumen of a lead. In some embodiments of the present invention, each tapered portion 348 is shaped so as to funnel the distal end of a stylet into guiding portion 350. When this is the case, tapered portion 348 and guiding portion 350 may facilitate insertion of the stylet into the lumen of a lead.

With reference to FIG. 7, it will be appreciated that guiding portion 350 and tapered portion 348 of each channel permit a portion of a lead to be received between first side 306 and second side 308 while a portion of a stylet is extending from a lumen of lead. The portion of the stylet extending from the lumen of the lead can extend through the guiding portions and the tapered portions of the channels. When this is the case, the stylet can be used to reposition the distal end of the lead, while, at the same time, testing connector 300 is used to make a plurality of electrical connections with the lead.

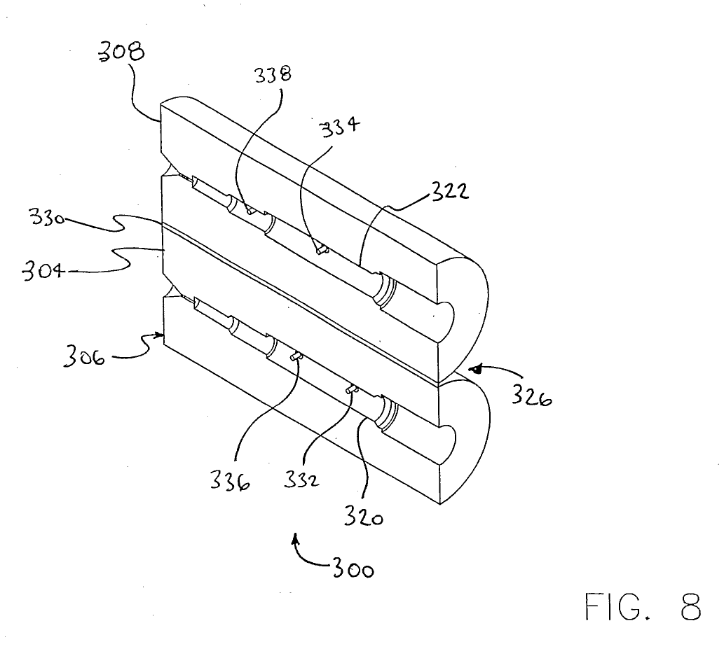

FIG. 8 is an additional isometric view of testing connector 300 shown in the previous figure. Testing connector 300 comprises a housing 304 including a first side 306 and a second side 308. In FIG. 8, first side 306 and second side 308 are hingingly coupled to one another by a hinge 326. In the embodiment of FIG. 8, hinge 326 comprises a web 330.

First side 306 of housing 304 defines a first channel 320 and second side 308 defines a second channel 322. In some embodiments of the present invention, the first side 306 and the second side 308 cooperate to define a cavity. In the embodiment of FIG. 8, first channel 320 and second channel 322 are shaped so that the cavity is capable of receiving a portion of a lead.

With reference to FIG. 8, it will be appreciated that first side 306 supports a first contact 332 and a third contact 336. In the embodiment of FIG. 8, first contact 332 and third contact 336 both extend into first channel 320. First contact 332 and third contact 336 may comprise, for example, spring-loaded contacts.

With continuing reference to FIG. 8, it will be appreciated that second side 308 of housing 304 defines a second channel 322. Second side 308 supports a second contact 334 and a fourth contact 338 that are shown extending into second channel 322. In some embodiments of the present invention, second contact 334 and fourth contact 338 are biased to extending into second channel 322. Second contact 334 and fourth contact 338 may comprise, for example, spring-loaded contacts.

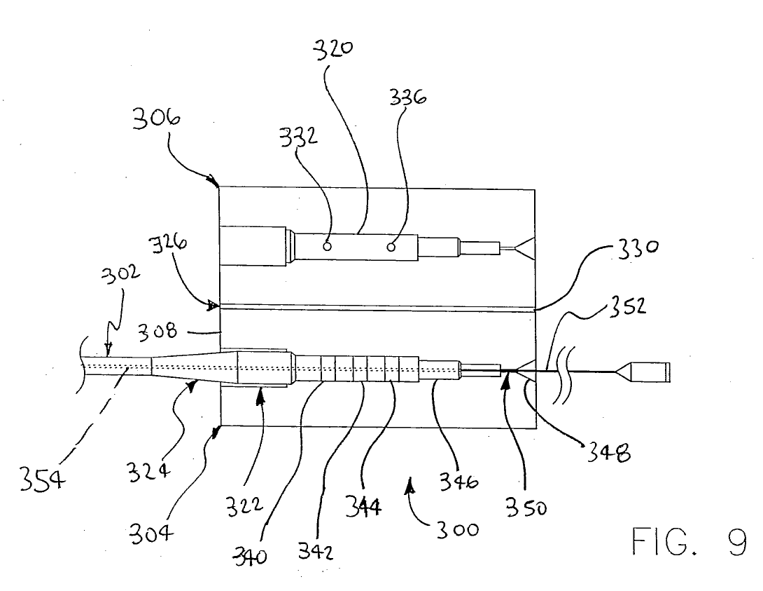

FIG. 9 is a plan view showing testing connector 300 shown in the previous figure. In the embodiment of FIG. 9, connector 324 of lead 302 is disposed in second channel 322 defined by second side 308 of housing 304. Stylet 352 can be seen extending from lumen 354 of lead 302. In FIG. 9, stylet 352 is shown extending through guiding portion 350 and tapered portion 348 of second channel 322.

Connector 324 of lead 302 comprises a first conductive ring 340, a second conductive ring 342, a third conductive ring 344, and a fourth conductive ring 346. A first side 306 of housing 304 is connected to second side 308 by a hinge 326. In the embodiment of FIG. 9, hinge 326 comprises a web 330.

With reference to FIG. 9, it will be appreciated that first side 306 supports a first contact 332 and a third contact 336. In the embodiment of FIG. 9, first contact 332 and third contact 336 both extend into first channel 320. In the embodiment of FIG. 9, first side 306 and second side 308 are disposed in an open configuration. Also in the embodiment of FIG. 9, first side 306 can be moved to a closed position in which a portion of connector 324 is disposed in first channel 320. When this is the case, first contact 332 may contact first conductive ring 340 and third contact 336 may contact third conductive ring 344. To facilitate the forming of a connection, first contact 332 and third contact 336 may be biased towards connector 324. For example, first contact 332 and third contact 336 comprise spring-loaded contacts.

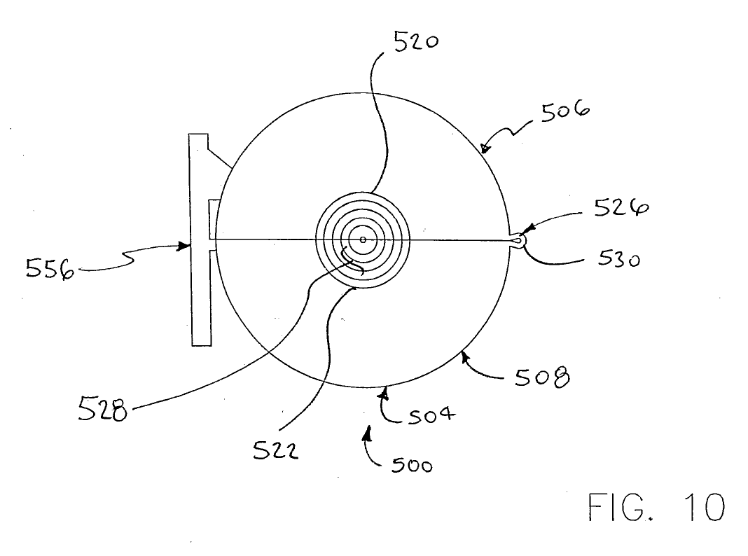

FIG. 10 is an axial plan view showing a testing connector 500 in accordance with an additional exemplary embodiment of the present invention. In the embodiment of FIG. 10, testing connector 500 comprises a housing 504. Housing 504 comprises a first side 506 that is connected to a second side 508 of housing 504 by a hinge 526. In the embodiment of FIG. 10, hinge 526 comprise a web 530. First side 506 defines a first channel 520 and second side 508 defines a second channel 522. In FIG. 10, first side 506 and second side 508 are disposed in a closed configuration so that first side 506 and second side 508 cooperate to define a cavity 528.

Second side 508 of housing 504 comprises a latch member 556. Latch member 556 is shown assuming a locking position in FIG. 10. In some methods in accordance with the present invention, latch member 556 is capable of holding first side 506 and second side 508 in the closed configuration shown in FIG. 10 while latch member 556 is assuming the locking position. Also in some methods in accordance with the present invention, latch member 556 is capable of assuming an unlocked position. When this is the case, first side 506 and second side 508 are free to assume an open configuration.

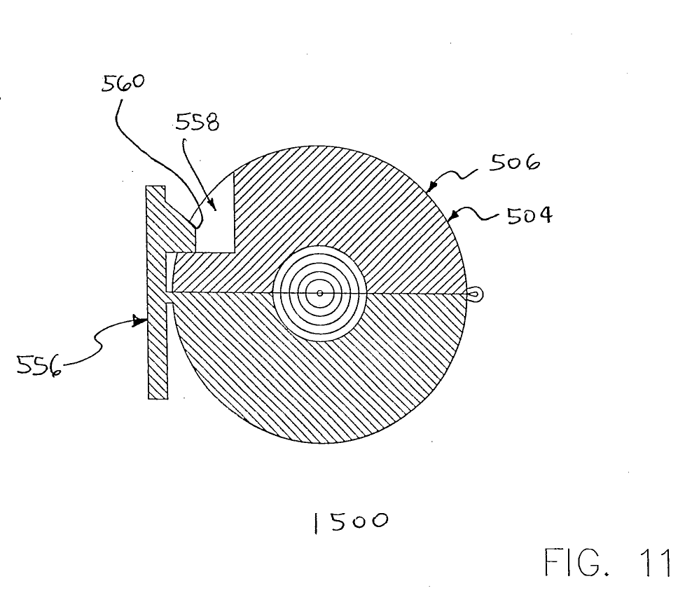

FIG. 11 is a cross-sectional view of the testing connector 500 shown in the previous figure. With reference to FIG. 11, it will be appreciated that first side 506 of housing 504 defines a depression 558 and second side 508 housing 504 comprises a latch member 556. In FIG. 11, a protrusion 560 of latch member 556 is shown extending into depression 558. The position of latch member 556 shown in FIG. 11 may be referred to as a locking position. In some methods in accordance with the present invention, latch member 556 is capable of holding first side 506 and second side 508 in a closed configuration while latch member 556 is in the locking position. Also in some methods in accordance with the present invention, latch member 556 is capable of assuming an unlocked position.

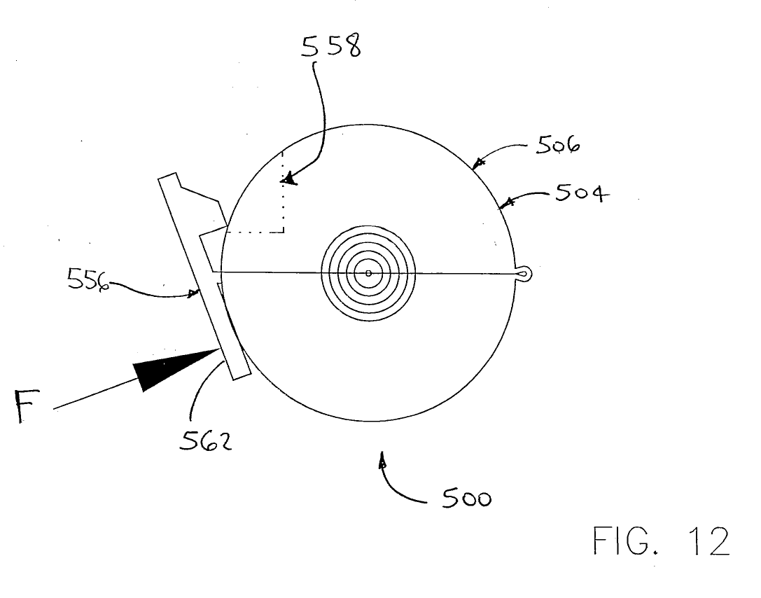

FIG. 12 is an additional axial view of testing connector 500 shown in the previous figure. In the embodiment of FIG. 12, a force F is shown acting on an actuating portion 562 of latch member 556. As shown in FIG. 12, force F has urged latch member 556 to assume an unlocked position in which protrusion 560 of latch member 556 is outside of depression 558 defined by first side 506 of housing 504. In some embodiments of the present invention, latch member 556 and housing 504 are dimensioned so that force F can be provided by the thumb of a hand while housing 504 is received in the palm of the hand.

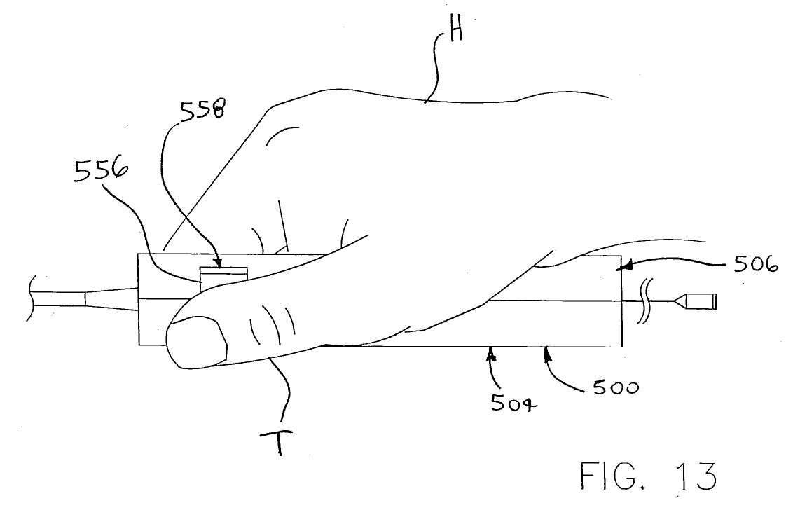

FIG. 13 is a side view including testing connector 500 shown in the previous figure. In the embodiment of FIG. 13, a hand H is disposed about housing 504 so that housing 504 is received in the palm of hand H. A thumb T of hand H is shown contacting latch member 556. Thumb T may be used to urge latch member 556 to assume an unlocked position in which the protrusion of latch member 556 is outside of depression 558 defined by first side 506 of housing 504.

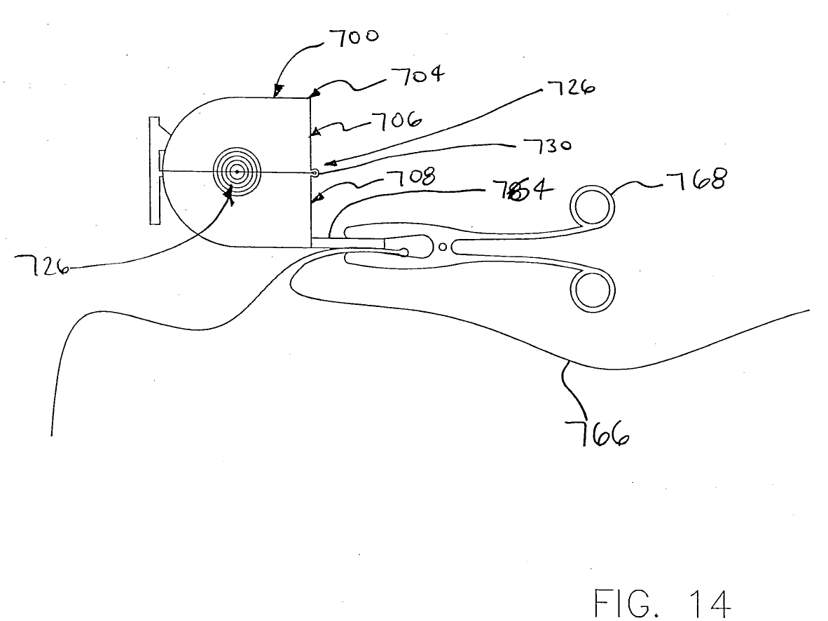

FIG. 14 is an axial plan view showing a testing connector 700 in accordance with yet another exemplary embodiment of the present invention. In the embodiment of FIG. 14, testing connector 700 comprises a housing 704 including a first side 706 and a second side 708. In the embodiment of FIG. 14, second side 708 comprises a tab 764. In the embodiment of FIG. 14, a sheet 766 is held against tab 764 by a clamp 768. Sheet 766 may comprise, for example, a surgical drape. Clamp 768 may comprise, for example, a surgical clamp.

In the embodiment of FIG. 14, second side 708 is connected to a first side 706 of housing 704 by a hinge 726. In the embodiment of FIG. 14, hinge 726 comprises a web 730. In FIG. 14, first side 706 and second side 708 are disposed in a closed configuration so that first side 706 and second side 708 cooperate to define a cavity 728. Cavity 728 may be dimensioned to receive a connector of an implantable lead.

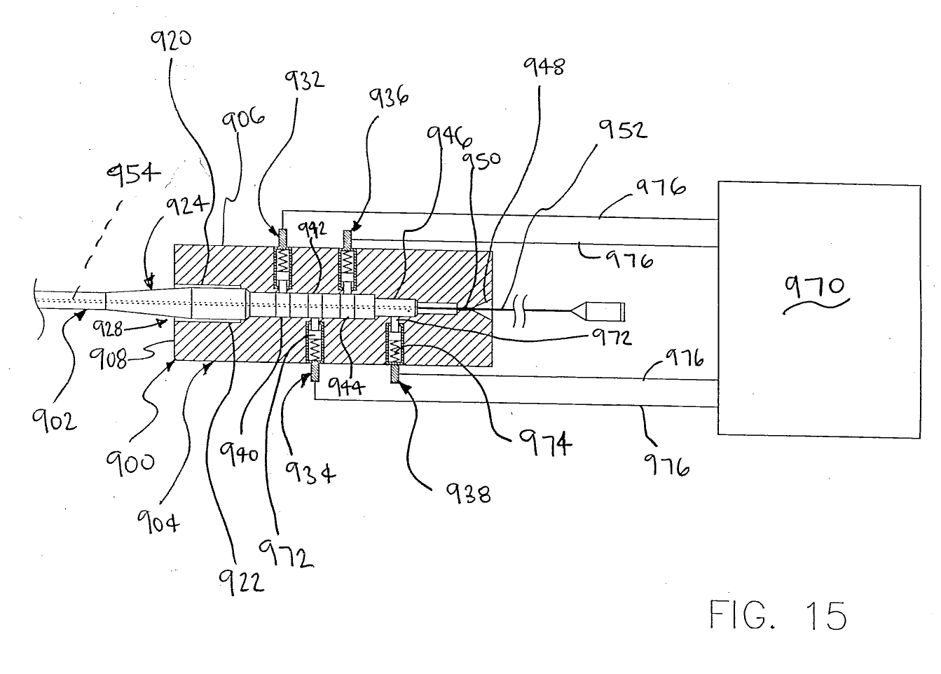

FIG. 15 is a cross-sectional view of an assembly including a testing connector 900 and an implantable lead 902. In some methods in accordance with the present invention, testing connector 900 may used to facilitate the making of electrical connection with implantable lead 902. Implantable lead 902 defines a lumen 954 that is dimensioned to receive a stylet 952. In some methods in accordance with the present invention, stylet 952 may be used to reposition the distal end of lead 902 within the body of a patient while lead 902 is electrically connected to an electronic device 970.

In the embodiment of FIG. 15, lead 902 includes a connector 924 comprising a first conductive ring 940, a second conductive ring 942, a third conductive ring 944, and a fourth conductive ring 946. Testing connector 900 comprises a housing 904 having a first side 906 and a second side 908. In the embodiment of FIG. 15, first side 906 of housing 904 defines a first hole and a third hole. In FIG. 15, a first contact 932 is shown disposed in the first hole. Also in FIG. 15, a third contact 936 is shown disposed in the third hole.

In the embodiment of FIG. 15, first side 906 defines a first channel 920 and second side 908 defines a second channel 922. In FIG. 15, connector 924 of lead 902 is shown partially disposed in first channel 920 and partially disposed in second channel 922. With continuing reference to FIG. 15, it will be appreciated that first side 906 and second side 908 of housing 904 cooperate to define a cavity 928. In the embodiment of FIG. 15, connector 924 of lead 902 is partially disposed in cavity 928. First contact 932 is shown contacting first conductive ring 940 of connector 924 of lead 902 in FIG. 15. Additionally, third contact 936 is shown contacting third conductive ring 944 in FIG. 15.

Second side 908 of housing 904 defines a second hole and a third hole. In FIG. 15, a second contact 934 is shown disposed in the second hole. Also in FIG. 15, a fourth contact 938 is shown disposed in the fourth hole. In the embodiment of FIG. 15, second contact 934 and fourth contact 938 comprise spring-loaded contacts. Also in the embodiment of FIG. 15, each spring-loaded contact comprises a contact tip 972 and a spring 974. In FIG. 15, the contact tip 972 of second contact 934 is shown contacting second conductive ring 942 of connector 924 of lead 902 in FIG. 15. Additionally, the contact tip 972 of fourth contact 938 is shown contacting fourth conductive ring 946 in FIG. 15.

In the embodiment of FIG. 15, a stylet 952 can be seen extending from lumen 954 of lead 902. In FIG. 15, stylet 952 can be seen extending through a guiding portion 950 of first channel 920 and a guiding portion 950 of second channel 922. Additionally, stylet 952 can be seen extending through a tapered portion 948 of first channel 920 and a tapered portion 948 of second channel 922 in FIG. 15. In the embodiment of FIG. 15, stylet 952 can be used to reposition the distal end of lead 902, while, at the same time, lead 902 is connected to electronic device 970 via testing connector 900.

In the embodiment of FIG. 15, first contact 932, second contact 934, third contact 936, and fourth contact 938 are all connected to electronic device 970 by wires 976. Electronic device 970 may comprise various elements without deviating from the spirit and scope of the present invention. Examples of electronic devices that may be suitable in some applications include defibrillator analyzers and pacemaker analyzers.

The housing of the testing connectors of the present invention can be composed of any non-conductive material suitable for use in medical procedures that does not damage the connector portion of the implantable lead. Suitable materials include homopolymers, copolymers, block copolymers and combinations thereof. Suitable polymers include, for example, polyethylene, polypropylene, poly(tetraflurorethylene), poly(vinylidene fluoride), poly(vinyl chloride), polyurethane, polycarbonate and blends and copolymers thereof. The electrically conductive contact members can be composed of any electrically conductive material, such as metals, metal alloys, conductive polymers, or combinations thereof. Suitable metals include nickel, aluminum, copper and combinations thereof. In some embodiments, the electrically conductive contact members can have a circular cross section, while in other embodiments the contact members may have an oval cross section, a rectangular cross section or the like. One of ordinary skill in the art will recognize that no particular cross sectional shape of the contact members is required by the present disclosure. The length of the contact members can be guided by the particular dimensions of the testing connector.

The housing portion of a preferred embodiment of the testing connector can be produced by any generally known plastic processing technique including, for example, extrusion, injection molding and compression molding. In some embodiments, the openings for the electrically conductive contact members can be formed integrally with the housing portion of the testing connector. In other embodiments, the openings for the contact members can be formed after the housing portion has been produced by, for example, drilling or the like. Generally, the electrically conductive contact members are inserted into the openings in the housing after formation of the housing.

The embodiments above are intended to be illustrative and not limiting. Additional embodiments are within the claims. Although the present invention has been described with reference to particular embodiments, workers in the art will recognize that changes may be made in form and detail without departing form the spirit and scope of the invention.

What is claimed is:

1. An apparatus comprising:
a housing comprising a first side and a second side;
the first side and the second side being movable relative to one another between a closed configuration and an open configuration;
the first side and the second side defining a cavity while the first side and the second side are deposed in the closed configuration;
the cavity being dimensioned so that the cavity is capable of receiving a portion of an implantable lead.

2. The apparatus of claim 1, further including a plurality of electrical contacts supported by the first side of the housing.

3. The apparatus of claim 2, wherein the contacts are axially spaced relative to one another.

4. The apparatus of claim 2, wherein a contact tip of each electrical contact is biased to extend into the cavity by a spring.

5. The apparatus of claim 1, wherein the first side of the housing defines a first channel.

6. The apparatus of claim 5, wherein the first channel comprises a guiding portion and a tapered portion.

7. The apparatus of claim 6, wherein the guiding portion of the first channel is positioned to be generally co-axially aligned with a lumen defined by an implantable lead when a portion of the implantable lead is positioned in the first channel.

8. The apparatus of claim 6, wherein the guiding portion of the first channel and the tapered portion of the first channel are dimensioned to allow a stylet to pass through the housing while a portion of an implantable lead is positioned in the first channel.

9. The apparatus of claim 1, wherein the second side of the housing defines a second channel.

10. The apparatus of claim 9, wherein the second channel comprises a guiding portion and a tapered portion.

11. The apparatus of claim 10, wherein the guiding portion of the second channel is positioned to be generally co-axially aligned with a lumen defined by an implantable lead when a portion of the implantable lead is positioned in the second channel.

12. The apparatus of claim 10, wherein the guiding portion of the second channel and the tapered portion of the second channel are dimensioned to allow a stylet to pass through the housing while a portion of an implantable lead is positioned in the second channel.

13. The apparatus of claim 1, further including a hinge connecting the first side of the housing to the second side of the housing.

14. The apparatus of claim 13, wherein the hinge comprises a web of polymeric material.

15. The apparatus of claim 1, further including a tab extending from the housing.

16. The apparatus of claim 15, further including a sheet held against the tab by a clamp.

17. The apparatus of claim 1, further including an implantable lead disposed in the cavity.

18. The apparatus of claim 17, further including a stylet extending through the housing and into a lumen defined by the implantable lead.

19. The apparatus of claim 18, further including an electronic device electrically connected to the lead by a plurality of wires.

20. The apparatus of claim 19, wherein the electronic device comprises a pacer analyzer.

21. The apparatus of claim 19, wherein the electronic device comprises a defibrillator analyzer.

22. An apparatus, comprising:
the housing comprising a first side and a second side;
a latch member coupled to the second side of the housing;
the first side of the housing defining a depression dimensioned to receive a protrusion of the latch member;
the first side defining a first channel;
the second side defining a second channel;
the first channel and the second channel being dimensioned to receive at least a portion of an implantable lead while at least a portion of the protrusion is disposed in the depression.

23. The apparatus of claim 22, wherein the latch member and the housing are dimensioned so that a thumb of a hand contacts the latch member while the housing is received in a palm of the hand.

24. The apparatus of claim 22, further including a hinge connecting the first side of the housing to the second side of the housing.

25. The apparatus of claim 24, wherein the hinge comprises a web of polymeric material.

26. The apparatus of claim 22, further including a first electrical contact supported by the first side of the housing.

27. The apparatus of claim 26, further including a first wire extending between the first contact an electronic device.

28. The apparatus of claim 27, wherein the electronic device comprises a pacer analyzer.

29. The apparatus of claim 27, wherein the electronic device comprises a defibrillator analyzer.

30. The apparatus of claim 22, further including a second electrical contact supported by the second side of the housing.

31. The apparatus of claim 30, further including a second wire extending between the second contact an electronic device.

32. The apparatus of claim 31, wherein the electronic device comprises a pacer analyzer.

33. The apparatus of claim 31, wherein the electronic device comprises a defibrillator analyzer.

34. The apparatus of claim 22, wherein the first channel comprises a guiding portion and a tapered portion.

35. The apparatus of claim 34, wherein the guiding portion of the first channel is positioned to be generally co-axially aligned with a lumen defined by an implantable lead when a portion of the implantable lead is positioned in the first channel.

36. The apparatus of claim 34, wherein the guiding portion of the first channel and the tapered portion of the first channel are dimensioned to allow a stylet to pass through the housing while a portion of an implantable lead is positioned in the first channel.

37. The apparatus of claim 22, wherein the second channel comprises a guiding portion and a tapered portion.

38. The apparatus of claim 37, wherein the guiding portion of the second channel is positioned to be generally co-axially aligned with a lumen defined by an implantable lead when a portion of the implantable lead is positioned in the second channel.

39. The apparatus of claim 37, wherein the guiding portion of the second channel and the tapered portion of the second channel are dimensioned to allow a stylet to pass through the housing while a portion of an implantable lead is positioned in the second channel.

40. The apparatus of claim 22, further including a tab extending from the housing.

41. The apparatus of claim 40, further including a sheet held against the tab by a clamp.

42. The apparatus of claim 22, further including an implantable lead disposed in the cavity.

43. The apparatus of claim 42, further including a stylet extending through the housing and into a lumen defined by the implantable lead.

44. An method, comprising the steps of:
providing an implantable lead comprising a connector;
electrically connecting an electronic device to at least one conductive surface of the connector of the lead;
inserting a stylet into a lumen defined by the implantable lead; and
repositioning a distal portion of the lead within a body while the lead is electrically connected to the electronic device.
